(19) United States
(12) Patent Application Publication
(10) Pub. No.: US 2025/0354137 A1
(43) Pub. Date: Nov. 20, 2025

(54) FIBRONECTIN LIBRARIES FOR HUMAN THERAPEUTIC SCREENING

(71) Applicants: Roberto CREA, Hillsborough, CA (US); Craig HOKANSON, Pleasanton, CA (US); Guido CAPPUCCILLI, Basel (CH); PROTELICA, INC., Hayward, CA (US)

(72) Inventors: Roberto CREA, Hillsborough, CA (US); Craig HOKANSON, Pleasanton, CA (US); Guido CAPPUCCILLI, Basel (CH)

(21) Appl. No.: 18/876,303

(22) PCT Filed: Jun. 20, 2023
(86) PCT No.: PCT/US2023/025724

§ 371 Date: Dec. 18, 2024

Related U.S. Application Data
(60) Provisional application No. 63/353,694, filed on Jun. 20, 2022.

(51) Int. Cl. C12N 15/10; C07K 14/78; C40B 40/10

(52) U.S. Cl. CPC C12N 15/1037; C07K 14/78; C40B 40/10

(57) ABSTRACT

Fibronectin libraries useful for efficient screening for specific binding proteins capable of binding a target at high affinity. The libraries, and the resulting binding proteins selected from the libraries, exhibit specific illustrated advantages.

### CROSS-REFERENCE TO RELATED APPLICATION

This application claims benefit of priority to U.S. Provisional Patent Application No. 63/353,694 filed on Jun. 20, 2022, which is incorporated herein by reference in its entirety.

### TECHNICAL FIELD

The present disclosure relates to the field of molecular biology and biological drug discovery. Fibronectin synthetic libraries are provided and are useful for efficient screening for specific binding proteins capable of binding a target entity with high affinity.

### BACKGROUND

Scaffold-based binding proteins are legitimate alternatives to antibodies in their ability to bind specific ligand targets. These scaffold binding proteins share the properties of having a stable framework core that can tolerate multiple substitutions in the ligand binding regions. Some scaffold frameworks have immunoglobulin-like protein domain architecture with loops extending from a β-sandwich core. A scaffold framework core can then be synthetically engineered from which a library of different sequence variants can be built upon. The sequence diversity is typically concentrated on the exterior surfaces of the proteins such as loop structures or other exterior surfaces that can serve as ligand binding regions.

Fibronectin Type III domains (FN3) were first identified as repeating domains in the fibronectin protein. The 14^{th} FN3 (14FN3) domain constitutes a small (about 89 amino acids), monomeric β-sandwich protein made up of seven β-strands with three connecting loops. The three “top” or three “bottom” loops of FN3 are functionally analogous to the complementarity-determining regions of immunoglobulin domains (see, e.g., U.S. Pat. Nos. 8,680,019 and 10,253,313, which are hereby incorporated by reference into this application). FN3 loop libraries can then be engineered to bind to a variety of targets such as cytokines, growth factors and receptor molecules. In addition, external targets on pathogens (e.g., viruses or bacteria) can be neutralized by such synthetic FN3 binding proteins.

One potential problem in creating FN3 libraries is the high frequency of unproductive variants leading to inefficient candidate screens. For example, creating diversity in the variants often involves in vitro techniques such as random mutagenesis, saturation mutagenesis, error-prone PCR, gene shuffling or walk-through mutagenesis. These strategies are inherently stochastic and often require the construction of exceedingly large libraries to comprehensively explore sufficient sequence diversity. Additionally, there is no way to enumerate the number, what type, and where in the protein the mutations have occurred. Furthermore, these random strategies create indiscriminate substitutions that destabilizes the FN3 or create unnatural amino acid sequences that may be rejected by the human immune system. It has been shown that improvement in one characteristic, such as affinity optimization, may also lead to decreased thermal stability when compared to the original protein scaffold framework.

Accordingly, a need still exists for FN3 libraries that are discrete in construction, excludes the use of mutagenesis, and maximizes diversity, while minimizing the occurrence of non-functional or truncated FN3 binding proteins. Such libraries benefit biologics drug discovery by lowering manpower and machine resources and decreasing time to drug candidate identification.

### SUMMARY

It has been discovered that a superior FN3-based library can be constructed without the use of any mutagenesis. Provided herein are libraries based on the fourteenth human fibronectin type III domain (14FN3). Binding proteins selected from the libraries can be further developed as biological drugs, e.g., for treating a condition or infection in humans. In general, the design, production, and use in drug discovery of FN3 libraries can be found in, for example, U.S. Pat. Nos. 8,680,019; 10,995,131; and 9,139,825.

These libraries are built upon the use of oligonucleotides with discretely defined sequences rather than relying on difficult-to-control mutagenesis. The tightly controlled variable amino acid sequences encoded by these libraries are designed to generally exclude non-conserved amino acid substitutions of the wild-type amino acid in the variable regions while allowing for ready replacement of only selected conserved amino acids, resulting in a library with maximal diversity for high affinity target binding and yet exhibiting minimal non-functional binding proteins due to nonsense, insertion, deletion, frameshift, duplication or other mutations. These libraries exclude amino acids at a particular position, whether conserved or not when compared to wild-type, within a variable region of a FN3 binding protein merely by ensuring no oligonucleotides encode such an amino acid.

In particular, the 14FN3 libraries encode wild-type regions A, AB, C, CD, D, E, EG and F, while top loops BC, DE, and FG are designed to be variable as further described below. The general secondary and tertiary structure of FN3 domains and locations of the various regions and loops can be found in, e.g., Sharma et al., EMBO J 18:1468-1479, 1999; and U.S. Pat. No. 8,680,019. FIG. 7 is a schematic diagram showing the β-strands and the six loops for human 14FN3.

The 14FN3 binding scaffold was selected from a number of naturally occurring FN3 sequences (see, e.g., U.S. Pat. No. 9,376,483) after a thorough bioinformatic analysis and stability comparison. In addition, any FN3 in general has several advantages over larger protein scaffolds, such as prototypical IgG monoclonal antibodies consisting of two binding sites and four polypeptide chains. Monomeric FN3 binding proteins are relatively small, less than 10% the size of a typical IgG, and therefore may access certain tissue compartments in vivo more easily than larger therapeutic molecules. In other circumstances, the therapeutic context may require or prefer a small, monomeric binding protein, rather than one that is multimeric, or where higher avidity is undesirable. From a manufacturing perspective, FN3-based drugs may avoid more expensive mammalian cell expression while taking advantage of lower cost bacterial, yeast or other microbial production systems, due to the lack of glycosylation sites and cysteines in FN3 scaffolds. There are other manufacturing advantages associated with the absence of the endogenous cysteines found in an antibody, particularly when present in a CDR, which can increase the redox complexity of antibody drug manufacturing processes, storage conditions and formulation development. This disadvantage for antibodies underscores the advantages in the FN3 libraries (e.g., 14FN3 libraries) described herein, as the oligonucleotides encoding the variable regions of FN3 exclude any cysteine residues as a general matter. FN3 binding proteins also tend to have superior thermostability and may be more easily formulated (e.g., because FN3 molecules can be more water soluble than IgG molecules) as compared to other proteins drugs. Finally, the highly conserved nature of natural FN3 proteins may offer superiorly low immunogenicity compared to IgG, because each position within a FN3 variable loop is discretely designed to maximize the amount of naturally conserved amino acid variability where possible. The higher level of conservation relative to natural FN3 sequences is expected to minimize immunogenicity compared to other, more immunogenic FN3 libraries.

The general advantages discussed immediately above does not exclude the possibility that a library designer may compromise one advantage to enrich the library in a different way. For example, if a cysteine is desired in either the variable region of a FN3 or in one of the otherwise wild-type β-strands (e.g., to facilitate covalent conjugation to a drug or other moiety), the library scaffold or the variable region can be discretely designed to incorporate that cysteine, even if the general FN3 (e.g., 14FN3) sequences may become less natural.

Accordingly, one embodiment described herein is a library of nucleic acids encoding a polypeptide having a non-natural 14th domain of human fibronectin type III (14FN3), the 14FN3 having (1) wild-type 14FN3 regions A, AB, B, C, CD, D, E, EF and F; (2) a loop BC described in SEQ ID NOs:1[FIG. 1, BC11], 2 [FIG. 2, BC14] or 3 [FIG. 3, BC 15]; (3) loop DE comprising SEQ ID NO:4 [FIG. 4, DE6]; and (4) loop FG comprising SEQ ID NOs:5[FIG. 5, FG8] or 6 [FIG. 6, FG11], where at least 90% (e.g., at least 95%, 96%, 97%, 98% or 99%) of the nucleic acids in the library encode the 14FN3. A library having such a high fidelity is possibly only because mutagenesis is avoided. Instead, discrete oligonucleotides are synthesized and used to build the library.

As noted above and further described below, the library is based on three different lengths of the variable BC loop (BC11, BC14 and BC15) and two different lengths of the variable FG loop (FG8 and FG11). There is only one length of the variable DE loop (DE6). All permutations of variable loop lengths for BC and FG, along with DE6, can be present in the library.

As shown for one specific library, which is detailed in the FIGS. 1 to 6, each position within a variable loop is varied in accordance with the priorities of the library designer. In this case, FIGS. 1 to 6 also show the target prevalence of each allowed residue at a particular variable loop position for this single library. While this library's variable amino acid prevalence was based on updated data regarding naturally occurring amino acids within fibronectin domains, as well as data on actual binding proteins selected from various FN3 libraries against a target antigen or epitope, other libraries may have different priorities and may exhibit different amino acid prevalences.

In this case at least 60% of the amino acids encoded at position 5 in BC14 is a P, at least 60% of the amino acids encoded at position 8 in BC15 is D, at least 60% of the amino acids encoded at position 10 in BC15 is G, at least 60% of the amino acids encoded at position 15 in BC15 is G, at least 60% of the amino acids encoded at position 6 in FG8 is S, at least 60% of the amino acids encoded at position 1 in FG 11 is N, at least 60% of the amino acids encoded at position 6 in FG11 is G and at least 60% of the amino acids encoded at position 9 in FG11 is S.

In other embodiments, the amino acid sequences of the variable loops in a 14FN3 library can be varied in accordance with any of the sequence rules herein, either singly, in combination or adhering to all rules at once, for any particular library. Other than the sequence rules above, the libraries may also follow these additional rules, particularly for a 14FN3 library: position 1 in BC11 is at least 85% hydrophilic, positive or negative; position 4 in BC11 is at least 50% conformational; position 5 in BC11 is at least 50% conformational; position 7 in BC11 is at least 60% G; position 9 in BC11 is at least 95% hydrophobic; position 10 in BC11 is at least 70% positive or negative; position 10 in BC11 is at least 65% negative; position 1 in BC14 is at least 83% hydrophilic, positive or negative; position 5 in BC14 is at least 95% conformational; position 6 in BC14 is at least 38% negative; position 7 in BC 14 is at least 46% negative; position 8 in BC14 is at least 50% conformational; position 10 in BC14 is at least 50% conformational; position 12 in BC14 is at least 90% hydrophobic; position 1 in BC15 is at least 58% hydrophilic; position 4 in BC15 is at least 50% conformational; position 5 in BC15 is at least 95% conformational; position 8 in BC15 is at least 69% negative; position 9 in BC15 is at least 95% conformational; position 10 in BC15 is at least 88% conformational; position 13 in BC15 is at least 95% hydrophobic; position 15 in BC15 is at least 61% G; position 5 in FG8 is at least 68% positive or negative; position 9 in FG8 is at least 95% hydrophilic, positive or negative; position 1 in FG11 is at least 95% hydrophilic, positive or negative; position 3 in FG11 is at least 64% hydrophobic; position 4 in FG11 is at least 95% G; position 6 in FG11 is at least 68% G; position 8 in FG11 is at least 56% P and position 9 in FG11 is at least 84% hydrophilic, positive or negative.

Consistent with modern molecular biology, the libraries disclosed herein can be a ribosome display library, a polysome display library, a phage display library, a bacterial expression library, or a yeast display library.

Also described are methods of identifying a binding protein having a desired binding affinity against a selected target or a desired function. These methods include the step of expressing the polypeptides from a library described herein, followed by the step of contacting the polypeptides expressed from the library with the selected target or substrate of a reaction and the step of either (1) screening for binding between one or more polypeptides of the library to the selected target, or (2) alternatively screening for a biochemical reaction involving the substrate. If the screen is designed to find library members that bind a target, the threshold binding can be expressed as having a K_{D} of less than about 1 nM (e.g., less than about 100 pM, 10 pM or 1 pM).

Also described herein are binding proteins identified by these screening methods. Such binding proteins can include a toxin covalently linked to the binding protein, or a multimer of the binding protein. In some cases, the multimer may be a dimer, such as a homodimer or a heterodimer. In general, the multimers can contain chains of the same FN3 (e.g., 14FN3) unit or different FN3 (e.g., 14FN3) units, each binding a different target, covalently linked together. Fusion protein linkers are known in the art and are described in, e.g., Chen et al., Adv Drug Deliv Rev, 65:1357-1369, 2013. Linkers between proteins and toxins are known in the art and are described in, e.g., Su et al., Acta Pharmaceutica Sinica B, 11:3889-3907, 2021. Also described herein is a chimeric antigen receptor including a binding protein described herein, a transmembrane domain, and an intracellular signaling domain.

The process of building the 14FN3 libraries described herein enable the generation of any FN3 library having three variable loops for binding to a target, while retaining the benefits of very high fidelity and high true diversity. Such FN3 libraries also exhibit many of the other advantages and benefits for 14FN3 libraries as described herein, without the requirement of the specific sequences shown in the FIGS. 1 to 6.

Accordingly, in one embodiment, a library of nucleic acids encoding a non-natural human fibronectin type III (FN3) is provided. This FN3 library contains wild-type FN3 (e.g., 14FN3 or 10FN3) regions A, B, C, D, E and F and three variable loops selected from AB, BC, CD, DE, EF and FG (e.g., variable loops BC, DE and FG), where the three variable loops, either at the top or bottom of the FN3, contacts and specifically binds a selected target at a K_{D} of at least 1 nM (e.g., at least 100 pM, 10 pM or 1 pM); and wild-type loops in the regions other than the three top or bottom variable loops. At least 90% (e.g., at least 95%, 98%, or 99%) of the nucleic acids in the library encode the FN3 and the nucleic acids encode at least about 10⁸ (e.g., at least about 10⁹, 10¹⁰, 10¹¹ or 10¹²) distinct functional FN3 species.

Unless defined otherwise, all technical and scientific terms used herein have the same meaning as commonly understood by those of ordinary skill in the art.

It is to be understood that both the foregoing general description and the following detailed description are exemplary and explanatory only and are not restrictive. Additional features will be set forth in part in the description which follows or may be learned by practice as described herein.

The foregoing and other features will become apparent to one skilled in the art upon consideration of the following description of exemplary embodiments.

### BRIEF DESCRIPTION OF THE DRAWINGS

The accompanying drawings, which are incorporated in and constitute a part of this specification, illustrate several embodiments and, together with the description, serve to explain the principles herein.

FIG. 1 is a table showing, in a representative library, the allowed amino acid sequence in a variable BC loop of length 11 amino acids (BC11), which is also designated as SEQ ID NO:1.

FIG. 2 is a table showing, in the same representative library as FIG. 1, the allowed amino acid sequence in variable BC loop of length 14 amino acids (BC14), which is also designated as SEQ ID NO:2.

FIG. 3 is a table showing, in the same representative library as FIG. 1, the allowed amino acid sequence in variable BC loop of length 15 amino acids (BC15), which is also designated as SEQ ID NO:3

FIG. 4 is a table showing, in the same representative library as FIG. 1, the allowed amino acid sequence in variable DE loop of length six amino acids (DE6), which is also designated as SEQ ID NO:4.

FIG. 5 is a table showing, in the same representative library as FIG. 1, the allowed amino acid sequence in variable FG loop of length eight amino acids (FG8), which is also designated as SEQ ID NO:5.

FIG. 6 is a table showing, in the same representative library as FIG. 1, the allowed amino acid sequence in variable FG loop of length 11 amino acids (FG11), which is also designated as SEQ ID NO:6.

FIG. 7 is a schematic diagram showing the amino acid sequence of wild-type human 14FN3 (SEQ ID NO: 7), with the amino acids in the loops or the ends of the domain shown in clear circles and the amino acids in the β-strands shown in greyed circles. Loops BC, DE and FG are labeled. Other FN3 domains and other loops on the “floor” of the molecule (e.g., the bottom loops) can also be modified as variable loops for use in the libraries described herein.

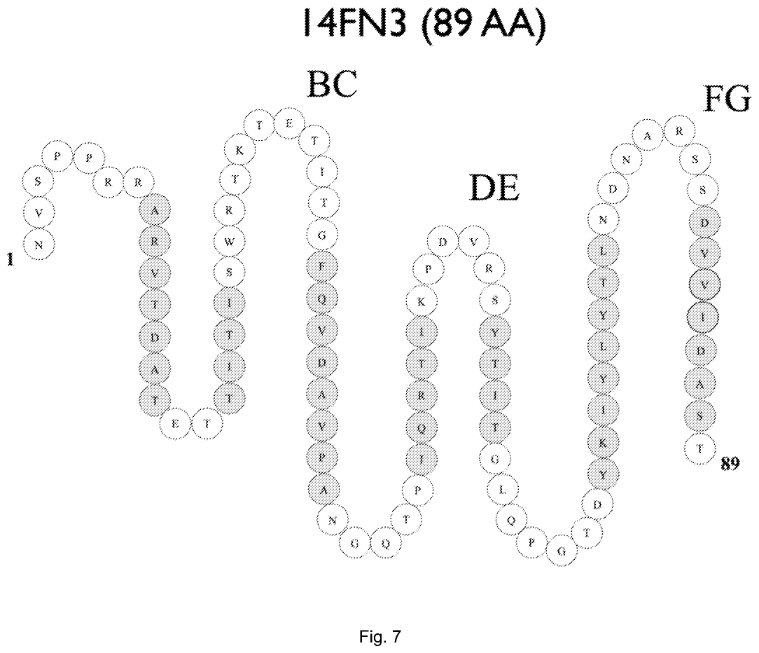

### DETAILED DESCRIPTION

Provided herein are improved FN3 libraries (e.g., 14FN3 libraries) that have high fidelity and diversity and can be screened to identify high affinity binding proteins against a target or screened for a particular function such as catalytic activity (see, e.g., the compendium Avalle et al., Chem Immunol vol. 77, 2000 titled “Catalytic Antibodies”).

As noted above, the FN3 libraries herein exhibit a very high fidelity in expressing full-length or functional binding proteins (e.g., greater than 90%). By “functional” in this library context is meant that the protein encoded by a member of the library is of full- length as intended by the designer of the library. Therefore, any noted diversity in such libraries is considered a true diversity in binding proteins, where the prevalence of truncated or spuriously mutated FN3 proteins is minimized. In other libraries, the diversity can be quite high, but that diversity includes a much larger prevalence of species which are not full-length or not functional, e.g., because such libraries are built on one of the many poorly-controlled mutagenic methods known in molecular biology. In that context, it is noted that the libraries herein can impressively encode and express a true diversity of at least about 10⁸ (e.g., at least about 10⁹, 10¹⁰, 10^{11,} or 10¹²) distinct functional FN3 species (e.g., 14FN3 species) using three variable loops (e.g., the top loops). In one embodiment, a 14FN3 library with variable top loops contains about 10¹² (e.g., about 1.3×10¹²) functional proteins.

Libraries constructed using oligonucleotides in the manner described herein might not achieve 100% fidelity due to spurious biochemical reactions during oligonucleotide synthesis or cloning. This is to be distinguished from and contrasted with the intentional mutagenesis that is performed in the construction of other libraries.

Within the context of a variable region of a FN3 library (e.g., a 14FN3 library), a “conserved” amino acid substitution is any substitution with an amino acid of the same amino acid group. These groups are “hydrophobic” (F, G, A, V, L, I, M), “aromatic” (W, Y), “hydrophilic” (S, T, Q, N,), “positive” (R, K, H), “negative” (D, E) and “conformational” (P, G). Of course, cysteine is typically not desirable in the variable region of an 14FN3 library designed for discovering protein drugs and therefore is not represented in the above grouping. In addition, a particular amino acid position within a variable region of an FN3 library may be designated as having an “hydrophobic” or “aromatic” or “hydrophilic” or “positive” or “negative” or “conformational” amino acid. In such a situation, the amino acid group name means that the position can contain any one of the amino acids in that group as defined above.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant host cell” (or simply “host cell”) or “cell line” refers to a cell into which a recombinant expression vector has been introduced. It is understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” or “cell line” as used herein.

The term “subject” includes humans and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably.

As used herein, the term “treating” or “treatment” of any disease or disorder (e.g., breast cancer) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, it is intended to include such other forms of expression vectors, such as viral vectors (e.g., replication defective retroviruses, adenoviruses and adeno-associated viruses), which serve equivalent functions.

The binding proteins selected from the libraries described herein can be conjugated with drugs to form FN3-drug conjugates (FDCs). Typically, the FDC contains a linker between the drug and the FN3. The linker can be a degradable or a non-degradable linker. Degradable linkers are typically easily degraded in the intracellular environment, for example, the linker is degraded at the target site, so that the drug is released from the FN3. Suitable degradable linkers include, for example, enzymatically degraded linkers, including peptidyl-containing linkers that can be degraded by intracellular proteases (such as lysosomal proteases or endosomal proteases), or sugar linkers, for example, a glucuronide-containing linker that can be degraded by glucuronidase. The peptidyl linker may include, for example, dipeptides such as valine-citrulline, phenylalanine-lysine or valine-alanine. Other suitable degradable linkers include, for example, pH-sensitive linkers (for example, linkers that are hydrolyzed at a pH of less than 5.5, such as hydrazone linkers) and linkers that degrade under reducing conditions (for example, disulfide bond linkers). Non-degradable linkers typically release the drug under conditions where the FN3 is hydrolyzed by a protease.

Before being connected to the FN3, the linker has a reactive group capable of reacting with certain amino acid residues, and the connection is achieved through the reactive group. Sulfhydryl-specific reactive groups include, for example, maleimide compounds, halogenated amides (such as iodine, bromine, or chloro); halogenated esters (such as iodine, bromine, or chloro); halogenated methyl ketones (such as iodine, bromine or chloro), benzyl halides (such as iodine, bromine or chloro); vinyl sulfone, pyridyl disulfide; mercury derivatives such as 3,6-Di-(mercury methyl) dioxane, and the counter ion is acetate, chloride or nitrate; and polymethylene dimethyl sulfide thiosulfonate. The linker may include, for example, maleimide linked to the FN3 via thiosuccinimide.

It is noted that the libraries described herein may exclude cysteine as an amino acid for reasons state elsewhere. However, in the case of drug conjugation, it may be desirable to insert a cysteine into an FN3 in any region of the protein, including in originally non-variable loops of the FN3 or in a portion of a β-strand of the FN3, particularly 14FN3.

The drug can be any cytotoxic, inhibiting cell growth or immunosuppressive drug. In embodiments, the linker connects the FN3 and the drug, and the drug has a functional group that can be bonded to the linker. For example, the drug may have an amino group, a carboxyl group, a sulfhydryl group, a hydroxyl group, or a ketone group that can form a bond with the linker. In the case where the drug is directly connected to the linker, the drug has a reactive group before being connected to the FN3.

Useful drug categories include, for example, anti-tubulin drugs, DNA minor groove binding reagents, DNA replication inhibitors, alkylating reagents, antibiotics, folate antagonists, antimetabolites, chemotherapy sensitizers, topoisomerase inhibitors, Vinca Alkaloids, etc. Typical cytotoxic drugs include, for example, auristatins, camptothecins, duocarmycins, etoposides, maytansines and maytansinoids (e.g., DM1 and DM4), taxanes, benzodiazepines or benzodiazepine containing drugs (e.g., pyrrolo[1,4] benzodiazepines (PBDs), indolinobenzodiazepines and oxazolidinobenzodiazepines and vinca alkaloids.

As described herein, the drug-linker can be used to form FDC in one simple step. In other embodiments, bifunctional linker compounds can be used to form FDCs in a two-step or multi-step process. For example, the cysteine residue reacts with the reactive part of the linker in the first step, and in the subsequent step, the functional group on the linker reacts with the drug to form FDC.

Generally, the functional group on the linker is selected to facilitate the specific reaction with the appropriate reactive group on the drug moiety. As a non-limiting example, the azide-based moiety can be used to specifically react with the reactive alkynyl group on the drug moiety. The drug is covalently bound to the linker through the 1,3-dipolar cycloaddition between the azide and alkynyl groups. Other useful functional groups include, for example, ketones and aldehydes (suitable for reacting with hydrazides and alkoxyamines), phosphines (suitable for reacting with azides); isocyanates and isothiocyanates (suitable for reaction with amines and alcohols); and activated esters, such as N-hydroxysuccinimide ester (suitable for reaction with amines and alcohols). These and other ligation strategies, such as those described in “Bioconjugation Technology”, Second Edition (Elsevier), are well known to those skilled in the art. Those skilled in the art can understand that for the selective reaction between the drug moiety and the linker, when a complementary pair of reactive functional groups is selected, each member of the complementary pair can be used for both linkers and drugs.

In one embodiment, the polynucleotides are engineered to serve as templates that can be expressed in a cell free extract. Vectors and extracts as described, for example in U.S. Pat. Nos. 5,324,637; 5,492,817; 5,665,563, can be used and many are commercially available. Ribosome display and other cell-free techniques for linking a polynucleotide (i.e., a genotype) to a polypeptide (i.e., a phenotype) can be used, e.g., Profusion™ (see, e.g., U.S. Pat. Nos. 6,348,315; 6,261,804; 6,258,558; and 6,214,553).

Alternatively, the polynucleotides can be expressed in a convenient E. coli expression system, such as that described by Pluckthun and Skerra. (Pluckthun, A. and Skerra, A., Meth. Enzymol. 178:476-515 (1989); Skerra, A. et al., Biotechnology 9:273-278 (1991)). The proteins can be expressed for secretion in the medium and/or in the cytoplasm of the bacteria, as described by M. Better and A. Horwitz, Meth. Enzymol. 178:476 (1989). In one embodiment, the fibronectin binding proteins are attached to the 3′ end of a sequence encoding a signal sequence, such as the ompA, phoA or pelB signal sequence (Lei, S. P. et al., J. Bacteriol. 169:4379 (1987)). These gene fusions are assembled in a bicistronic construct, so that they can be expressed from a single vector and secreted into the periplasmic space of E. coli where they will refold and can be recovered in active form. (Skerra, A. et al., Biotechnology 9:273-278 (1991)).

In another embodiment, the fibronectin binding domain sequences are expressed on the membrane surface of a prokaryote, e.g., E. coli, using a secretion signal and lipidation moiety as described, e.g., in U.S. Pat. No. 20040072740A1; U.S. Pat. No. 20030100023A1; and U.S. Pat. No. 20030036092A1.

In still another embodiment, the polynucleotides can be expressed in eukaryotic cells such as yeast using, for example, yeast display as described, e.g., in U.S. Pat. Nos. 6,423,538; 6,331,391; and 6,300,065. In this approach, the fibronectin binding domain molecules of the library are fused to a polypeptide that is expressed and displayed on the surface of the yeast.

Higher eukaryotic cells for expression of the fibronectin binding proteins can also be used, such as mammalian cells, for example myeloma cells (e.g., NS/0 cells), hybridoma cells, or Chinese hamster ovary (CHO) cells. Typically, the fibronectin binding domain molecules when expressed in mammalian cells are designed to be secreted into the culture medium or expressed on the surface of such a cell. The fibronectin binding domain can be produced, for example, as single individual module or as multimeric chains comprising dimers, trimers, that can be composed of the same module or of different module types.

The screening of the expressed fibronectin binding domain (or fibronectin binding domain produced by direct synthesis) can be done by any appropriate means. For example, binding activity can be evaluated by standard immunoassay and/or affinity chromatography. Screening of the fibronectin binding protein for catalytic function, e.g., proteolytic function can be accomplished using a standard hemoglobin plaque assay as described, for example, in U.S. Pat. No. 5,798,208. Determining the ability of candidate fibronectin binding domain to bind therapeutic targets can be assayed in vitro using, e.g., a Biacore instrument, which measures binding rates of a fibronectin binding domain to a given target or ligand. In vivo assays can be conducted using any of a number of animal models and then subsequently tested, as appropriate, in humans.

This disclosed may be further illustrated by the embodiments described in the following items:

Item 1 A library of nucleic acids encoding a polypeptide comprising a non-natural 14th domain of human fibronectin type III (14FN3), the non-natural 14FN3 comprising
a. wild-type 14FN3 regions A, AB, B, C, CD, D, E, EF and F, wherein the regions A, AB, B, C, CD, D, E, EF and F are arranged in the same order as they are arranged in wild-type 14FN3;
b. loop BC comprising one polypeptide selected from the group consisting of SEQ ID NOs:1, 2 and 3, wherein loop BC is positioned between regions B and C;
c. loop DE comprising SEQ ID NO:4, wherein loop DE is positioned between regions D and E; and
d. loop FG comprising one polypeptide selected from the group consisting of SEQ ID NOs:5 and 6, wherein loop FG is positioned after region F,
   wherein at least 90% of the nucleic acids in the library encode the 14FN3.

Item 2 The library of Item 1, wherein at least 95% of the nucleic acids in the library encode the 14FN3.

Item 3 The library of any one of the preceding items, wherein at least 99% of the nucleic acids in the library encode the 14FN3.

Item 4 The library of any one of the preceding items, wherein loop BC comprises SEQ ID NO:1.

Item 5 The library of any one of items 1-3, wherein loop BC comprises SEQ ID NO:2.

Item 6 The library of any one of items 1-3, wherein loop BC comprises SEQ ID NO:3.

Item 7 The library of any one of items 1-6, wherein loop FG comprises SEQ ID NO:5.

Item 8 The library of any one of items 1-6, wherein loop FG comprises SEQ ID NO:6.

Item 9 The library of any one of items 1-8, wherein at least 60% of the amino acids encoded at position 5 in loop BC is a P.

Item 10 The library of any one of items 1-9, wherein at least 60% of the amino acids encoded at position 8 in loop BC is D.

Item 11 The library of any one of items 1-10, wherein at least 60% of the amino acids encoded at position 10 in loop BC is G.

Item 12 The library of any one of items 1-11, wherein at least 60% of the amino acids encoded at position 15 in loop BC is G.

Item 13 The library of any one of items 1-12, wherein at least 60% of the amino acids encoded at position 6 in loop FG is S.

Item 14 The library of any one of items 1-13, wherein at least 60% of the amino acids encoded at position 1 in loop FG is N.

Item 15 The library of any one of items 1-14, wherein at least 60% of the amino acids encoded at position 6 in loop FG is G.

Item 16 The library of any one of items 1-15, wherein at least 60% of the amino acids encoded at position 9 in loop FG is S.

Item 17 The library of any one of items 1-16, wherein the nucleic acids encode at least about 10⁸ distinct functional 14FN3 species.

Item 18 The library of any one of items 1-17, wherein the nucleic acids encode at least about 10¹⁰ distinct functional 14FN3 species.

Item 19 The library of any one of items 1-18, wherein the nucleic acids encode at least about 10¹¹ distinct functional 14FN3 species.

Item 20 The library of any one of items 1-19, wherein the nucleic acids encode about 10¹² distinct functional 14FN3 species.

Item 21 The library of any one of items 1-20, wherein the library is an expression library selected from the group consisting of a ribosome display library, a polysome display library, a phage display library, a bacterial expression library, and a yeast display library.

Item 22 A method of identifying a binding protein having a desired binding affinity against a selected target, comprising
a. expressing the polypeptides from the library of any one of items 1-21;
b. contacting the polypeptides with the selected target; and
c. screening for binding between one or more polypeptides of the library to the selected target.

Item 23 The method of item 22, wherein the screening step further comprises determining that the binding achieves a K_{D} of at least 1 nM.

Item 24 The method of any one of items 22-23, wherein the screening step further comprises determining that the binding achieves a K_{D} of at least 1 pM.

Item 25 A binding protein identified by the method any one of items 22-24.

Item 26 The binding protein of Item 25, further comprising a toxin covalently linked to the binding protein.

Item 27 A multimer of the binding protein of any one of items 25-26.

Item 28 The multimer of Item 27, wherein at least two monomers are covalently linked.

Item 29 A chimeric antigen receptor comprising the binding protein of any one of items 25-26, a transmembrane domain, and an intracellular signaling domain.

Item 30 A library of nucleic acids encoding a polypeptide comprising a non-natural human fibronectin type III (FN3), the FN3 comprising
a. wild-type FN3 regions A, B, C, D, E and F
b. three variable loops selected from the group consisting of AB, BC, CD, DE, EF and FG, wherein the three variable loops contacts and specifically binds a selected target at a K_{D} of at least 1 nM; and
c. wild-type loops in the regions other than the three variable loops, wherein at least 90% of the nucleic acids in the library encode the FN3 and the nucleic acids encode at least about 10⁸ distinct functional FN3 species.

Item 31 The library of Item 30, wherein at least 95% of the nucleic acids in the library encode the FN3.

Item 32 The library of any one of items 30-31, wherein at least 99% of the nucleic acids in the library encode the FN3.

Item 33 The library of any one of items 30-32, wherein the nucleic acids encode at least about 10¹⁰ distinct functional FN3 species.

Item 34 The library of any one of items 30-33, wherein the nucleic acids encode at least about 10¹² distinct functional FN3 species.

Item 35 The library of any one of items 30-34, wherein the FN3 is a 14FN3.

Item 36 The library of any one of items 30-35, wherein the three variable loops are BC, DE and FG.

Item 37 The library of any one of items 1-21, wherein the non-natural 14FN3 further comprises a wild-type 14FN3 region G, and wherein the loop FG is positioned between regions F and G.

The following are examples of making and using a FN3 library.

### Example 1: Design of an 14FN3 Library with Variable BC, DE and FG Loops

A 14FN3 fibronectin loop library was constructed with the benefit of updated sequence and structural information to obtain an improved library. Structural molecular replacement modeling information was used to guide the selection of amino acid diversity to be introduced into variable loops BC, DE and FG. Still further, actual results obtained from selected fibronectin binding domains against specific targets helped guide the inclusion or exclusion of specific amino acids at positions within variable loops BC, DE and FG. For details regarding the design of FN3 libraries see, e.g., U.S. Pat. No. 8,680,019.

With the advantage of updated data regarding FN3 libraries, an improved 14FN3 library was constructed with three variable loops: BC, DE and FG (the top loops). All other regions of 14FN3 in the library were designed using wild-type sequences. Instead of using any mutagenic processes for the sequence variability, specific oligonucleotides were synthesized and bulk cloned, and inserted into 14FN3 expression vectors. This allows precise control of the amino acid sequence variability and a desired prevalence of each amino acid residue at each position; the higher the prevalence desired, the more oligonucleotides encoding that amino acid is present in the bulk cloning.

The 14FN3 three-loop library was built by assembling synthesized oligonucleotide pools of the BC, DE, and FG loops into the phagemid vector PADL-22c. The M13 attachment protein G3P was fused to the c-terminus of the 14FN3. In total, 36,000 oligonucleotides were printed into three pools. For the BC loop, there were 18,000 oligonucleotides because the BC loop has three lengths. For the DE loop, there were 6,000 oligonucleotides, and for the FG loop, there were 12,000 oligonucleotides for the two lengths. The length of the oligos were around 100 base pairs, so each 14FN3 was assembled with only three oligonucleotides. A total of 10 billion unique variants were cloned to form the library. The three-loop library is designed for phage display. The cloned library is transformed into TG1 cells, and phage particles were amplified and purified into a concentrated phage stock.

There were no silent mutations in the oligonucleotides for the library construction. The oligonucleotides for each loop were printed in sets of 6,000 with the frequency at each position inputted into the program running the oligonucleotide synthesizer. All the oligos were codon optimized for bacterial expression. For clarity, degenerate oligonucleotides containing equimolar amounts of different bases at a position were not used. The 36,000 oligos for the library are precisely synthesize according to the library design. Thus, the final library is a random sampling of the theoretical sequence space.

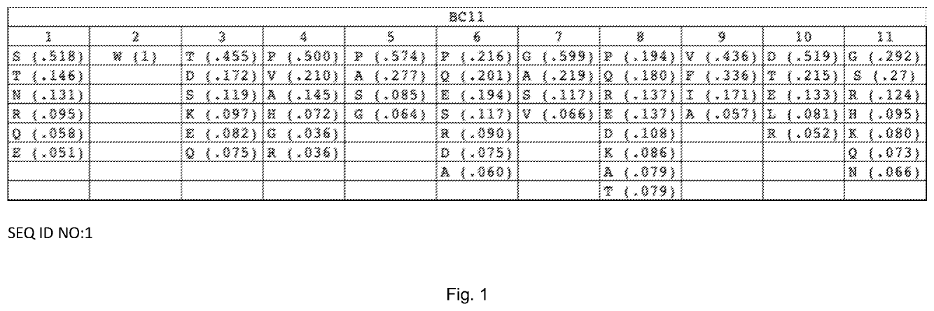

FIG. 1 is a table that shows the design of the BC loop in the library. Each column lists the allowed amino acid at that position in the loop, while the numerical value in parenthesis represents the target prevalence of the amino acid at that position. The prevalence of any particular amino acid was controlled by adding differential amounts of the oligonucleotides responsible for encoding the particular amino acid versus the amounts of oligonucleotide encoding other amino acids.

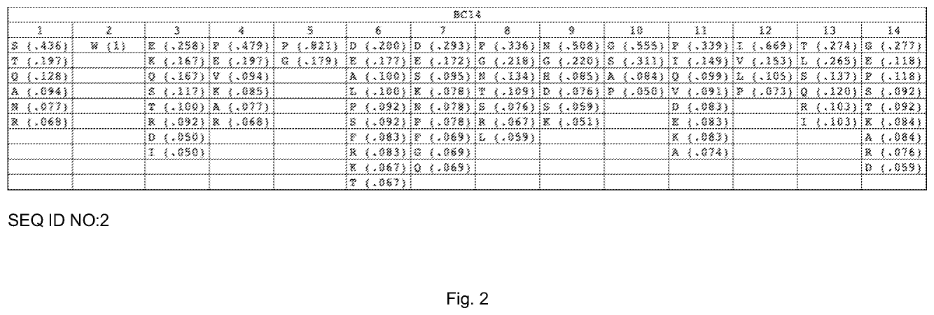

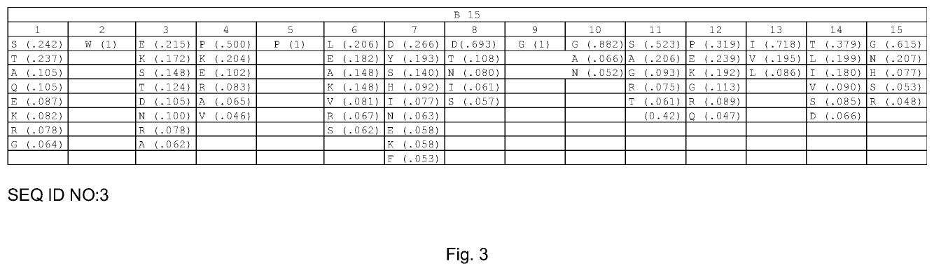

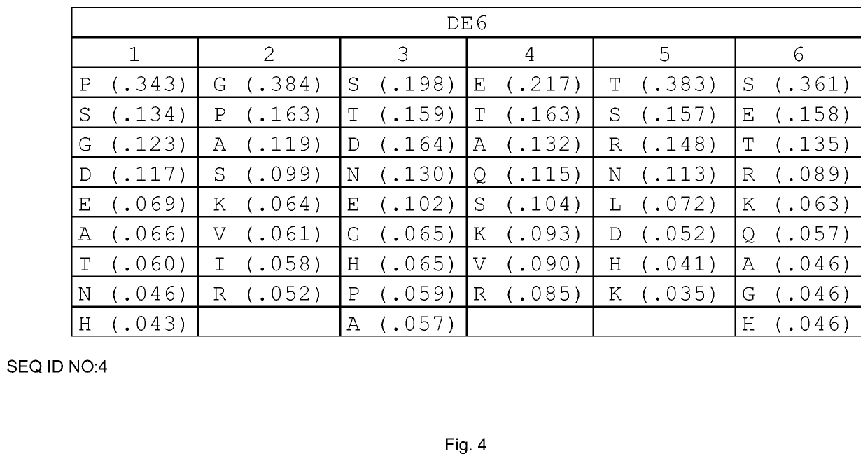

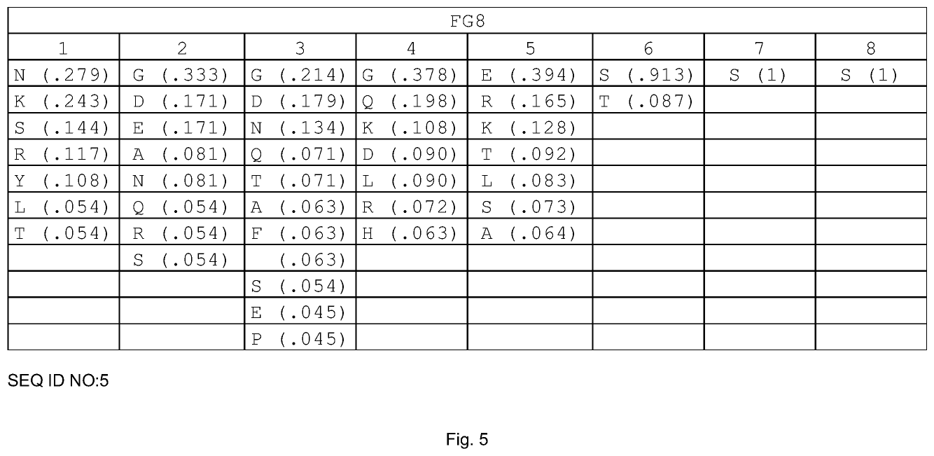

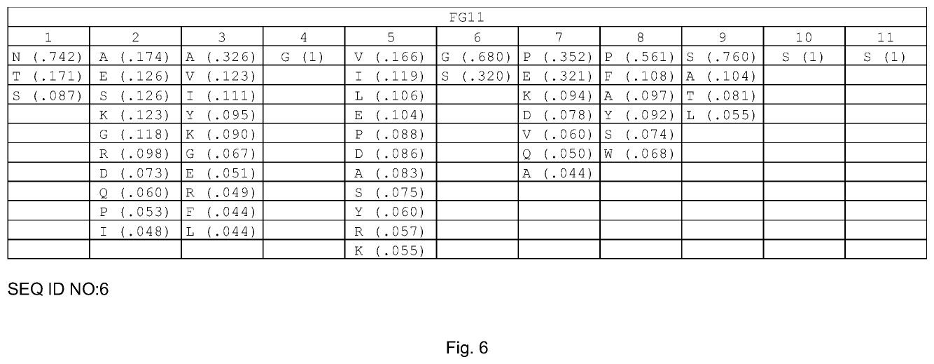

Similar to BC11, the sequence variability for all variable loops at all allowed lengths for this library are shown in FIGS. 1 to 6.

The library was validated using Next Generation Sequencing. Comparing the expected sequence variation with the observed sequence variation in the library, it was determined that about 95% of the library encoded full-length 14FN3 proteins.

### Example 2: Use of the Library in Example 1 for Discovery of Candidate Drug

The library of Example 1 was screened by phage display against the spike protein of a human pathogenic virus. A variant library with about 10¹⁰ true diversity was amplified in TG1 cells, and a concentrated phage stock purified. Four rounds of bead-based bio-panning was completed with decreasing target protein concentrations and increasing wash stringency. After phage ELISAs and sequencing, 46 unique 14FN3 clones that specifically bound to the spike protein were selected. For convenience, these clones were designated as 14FN3sp proteins. The nucleic acids encoding these 46 14FN3sp proteins were cloned in-frame into an IgG1 fusion cassette in a CMV-driven mammalian expression vector. The 14FN3sp clones were expressed in an HEK-293 cell expression system. The proteins were purified by a protein A resin, and the binding affinities of each 14FN3sp protein were determined by Carterra® SPR Kinetic Analysis. The best binding clone exhibited an affinity against the target at a K_{D} of 27 pM, while several other 14FN3sp clones exhibited low nanomolar binding to the target. CE-SDS electropherograms were used to quantitate the purified protein, and HPLC was run on the samples to look for aggregation in the preps. An UNCLE protein stability screening platform calculated the melting temperatures (Tm) of the 46 14FN3sp proteins. The 14FN3sp protein having a K_{D} of 27 pM against the target spike protein became the lead candidate for further drug development.

1. A library of nucleic acids encoding a polypeptide comprising a non-natural 14th domain of human fibronectin type III (14FN3), the non-natural 14FN3 comprising a. wild-type 14FN3 regions A, AB, B, C, CD, D, E, EF and F, wherein the regions A, AB, B, C, CD, D, E, EF and F are arranged in the same order as they are arranged in wild-type 14FN3;
b. loop BC comprising one polypeptide selected from the group consisting of SEQ ID NOs:1, 2 and 3, wherein loop BC is positioned between regions B and C;
c. loop DE comprising SEQ ID NO:4, wherein loop DE is positioned between regions D and E; and
d. loop FG comprising one polypeptide selected from the group consisting of SEQ ID NOs:5 and 6, wherein loop FG is positioned after region F, wherein at least 90% of the nucleic acids in the library encode the 14FN3.

2. The library of claim 1, wherein at least 95% of the nucleic acids in the library encode the 14FN3.

3. The library of claim 1, wherein at least 99% of the nucleic acids in the library encode the 14FN3.

4. The library of claim 1, wherein loop BC comprises SEQ ID NO:1 .

5. The library of claim 1, wherein loop BC comprises SEQ ID NO:2.

6. The library of claim 1, wherein loop BC comprises SEQ ID NO:3.

7. The library of claim 1, wherein loop FG comprises SEQ ID NO:5.

8. The library of claim 1, wherein loop FG comprises SEQ ID NO:6.

9. The library of claim 5, wherein at least 60% of the amino acids encoded at position 5 in loop BC is a P.

10. The library of claim 6, wherein at least 60% of the amino acids encoded at position 8 in loop BC is D.

11. The library of claim 6, wherein at least 60% of the amino acids encoded at position 10 in loop BC is G.

12. The library of claim 6, wherein at least 60% of the amino acids encoded at position 15 in loop BC is G.

13. The library of claim 7, wherein at least 60% of the amino acids encoded at position 6 in loop FG is S.

14. The library of claim 8, wherein at least 60% of the amino acids encoded at position 1 in loop FG is N.

15. The library of claim 8, wherein at least 60% of the amino acids encoded at position 6 in loop FG is G.

16. The library of claim 8, wherein at least 60% of the amino acids encoded at position 9 in loop FG is S.

17. The library of claim 1, wherein the nucleic acids encode at least about 10⁸ distinct functional 14FN3 species.

18. The library of claims 1, wherein the nucleic acids encode at least about 10¹⁰ distinct functional 14FN3 species.

19. The library of claim 1, wherein the nucleic acids encode at least about 10¹¹ distinct functional 14FN3 species.

20. The library of claims 1, wherein the nucleic acids encode about 10¹² distinct functional 14FN3 species.

21. The library of claim 1, wherein the library is an expression library selected from the group consisting of a ribosome display library, a polysome display library, a phage display library, a bacterial expression library, and a yeast display library.

22. A method of identifying a binding protein having a desired binding affinity against a selected target, comprising a. expressing the polypeptides from the library of claim 1;
b. contacting the polypeptides with the selected target; and
c. screening for binding between one or more polypeptides of the library to the selected target.

23. The method of claim 22, wherein the screening step further comprises determining that the binding achieves a K_{D} of at least 1 nM.

24. The method of claim 22, wherein the screening step further comprises determining that the binding achieves a K_{D} of at least 1 pM.

25. A binding protein identified by the method of claim 22.

26. The binding protein of claim 25, further comprising a toxin covalently linked to the binding protein.

27. A multimer of the binding protein of claim 25.

28. The multimer of claim 27, wherein at least two monomers are covalently linked.

29. A chimeric antigen receptor comprising the binding protein of claim 25, a transmembrane domain, and an intracellular signaling domain.

30. A library of nucleic acids encoding a polypeptide comprising a non-natural human fibronectin type III (FN3), the FN3 comprising
a. wild-type FN3 regions A, B, C, D, E and F
b. three variable loops selected from the group consisting of AB, BC, CD, DE, EF and FG, wherein the three variable loops contacts and specifically binds a selected target at a K_{D} of at least 1 nM; and
c. wild-type loops in the regions other than the three variable loops, wherein at least 90% of the nucleic acids in the library encode the FN3 and the nucleic acids encode at least about 108 distinct functional FN3 species.

31. The library of claim 30, wherein at least 95% of the nucleic acids in the library encode the FN3.

32. The library of claim 31, wherein at least 99% of the nucleic acids in the library encode the FN3.

33. The library of claim 30, wherein the nucleic acids encode at least about 10¹⁰ distinct functional FN3 species.

34. The library of claim 30, wherein the nucleic acids encode at least about 10¹² distinct functional FN3 species.

35. The library of claim 30, wherein the FN3 is a 14FN3.

36. The library of claim 35, wherein the three variable loops are BC, DE and FG.

37. The library of claim 1, wherein the non-natural 14FN3 further comprises a wild-type 14FN3 region G, and wherein the loop FG is positioned between regions F and G.
